(19) United States
(12) Patent Application Publication
(10) Pub. No.: US 2010/0004157 A1
(43) Pub. Date: Jan. 7, 2010

(54) PHARMACEUTICAL COMPOSITIONS FOR THE ORAL OR RECTAL ADMINISTRATION OF PROTEIN SUBSTANCES

(75) Inventors: Mauro Ajani, Lainate (Milano) (IT); Luigi Moro, Lainate (Milano) (IT); Roberto Villa, Lecco (IT)

Correspondence Address: DARBY & DARBY P.C., P.O. BOX 770, Church Street Station, New York, NY, 10008-0770, US

(73) Assignee: COSMO TECHNOLOGIES LTD., Dublin (IE)

(21) Appl. No.: 12/441,112

(22) PCT Filed: Sep. 7, 2007
(86) PCT No.: PCT/EP07/59378

§ 371 Date: Mar. 12, 2009

(30) Foreign Application Priority Data
Sep. 12, 2006 (IT) MI2006A001741
Jun. 15, 2007 (IT) MI2007A001205

(51) Int. Cl. A61K 38/00; A61P 1/00

(52) U.S. Cl. 514/2

(57) ABSTRACT

Pharmaceutical compositions with differentiated, controlled and/or site-specific release are claimed for the oral or rectal administration of peptide or protein substances, including antibodies and soluble receptors capable of antagonising the pathogenetic role of several cell mediators such as interleukines, chemokines, growth factors, tissue necrosis factors, and interferons. Through the incorporation of the peptide or protein substance inside a controlled and/or site-specific release preparation, the application of this invention permits transporting the substances directly into the intestinal environment where a reduced quantity of proteolytic enzymes is present, a less aggressive microenvironment for the integrity of the protein structure and sequence.

The present invention regards pharmaceutical compositions with differentiated, controlled and/or site-specific release, and more specifically for the administration of active principles of protein or polypeptide nature.

It is known how intercellular communications is entrusted to several chemical mediators that the cells specifically produce when they are subjected to particular stress or exposed to several particular conditions. Among these mediators, the following take on particular importance: interleukins [IL], chemokines [CM], growth factors [GF], interferons [IFN] and tumour necrosis factors [TNF]. The appearance/disappearance of some of these or a modification of their normal concentration level can constitute a factor which starts an immune response, or can represent an important sign of an event about to occur.

It is in fact known that these chemical mediators take on particular importance in inflammatory phenomena, in which they have the capacity to modulate in a positive and repressive sense both the etiology of the phenomenon and the organism's response, with the activation of reparation activities.

The TNF family can be indicated among the cytokines most used by the organism for modulating and controlling inflammatory and immune phenomenon.

These cytokines have shown to be particularly important in inducing inflammatory states, and their contribution is particularly fundamental in the tissues which are well supplied with dedicated receptors.

In the scope of the intestinal pathologies, for example, a high increase of TNF was found with the appearance of symptoms tied to inflammatory pathologies, in particular intestinal inflammatory pathologies (IBD), such as for example ulcerative colitis [UC] or Crohn's disease [CD]. The importance of TNF in the control of these inflammatory phenomena is so evident that immunomodulatory drugs have been directed on these pathologies, drugs which are especially designed to block the excess circulating TNF which can come into contact with the related specific receptors, positioned at the level of the cell membrane.

Starting from the beginning of the 1990s, in fact, protein substances appeared in the pharmaceutical repertory capable of blocking the specific TNF or its receptor sites, and therefore preventing the intracellular transmission phenomena that this contact gives rise to. It is in fact known that tumour necrosis factors, both of alpha and beta type, interact with the transmembrane receptor structures, already identified and in turn known: one, identified as P55, is a protein having 55 kDaltons molecular weight and set to translate signals which produce the classic cytotoxic, antiviral and proliferative effects of the TNFs. The other structure is known as P75; it is more indicated as TNFα receptor, and is a glycosylated protein which, in addition to the preceding signals, also produces an increase of the GM-CSF cell secretion.

These receptor structures are present on the cell membrane and have the function, once the receptor site is engaged with the suitable specific substrate, of activating transcriptional inputs inside the cell with the intracellular part of the receptor protein complex, which is clearly differentiated for TNFα and TNFβ.

Indeed, the use of imitation receptor protein structures is based on this recognised cell surface of the receptor sites capable of forming bonds with the TNFs. Such imitation reception protein structures are suitably synthesised or isolated, of similar composition but with reduced sequence and soluble nature, capable of binding with the circulating TNFα, preventing it from forming the pharmacodynamically important bonds with its real receptor positioned on the cell membrane.

Aderka et al. in 1992 published a work on Isrl. Med. Sci., 28, 126-130, where they described these soluble receptor structures and their capacity to form bonds with the specific cytokine. In such a manner, it was possible to attribute these soluble receptor structures with the capacity to act as part of the negative feed-back mechanism, set to control the in vivo activity of TNFα.

Wallach et al, in EP 526 905, describe this use for multimeric structures of soluble receptor form of the TNF containing portions of the P55 structure, produced both synthetically and by recombinant technology, useful for protecting human organisms from the deleterious effects of excess circulating cytokine.

The comprehension of this mechanism has opened the door to the possibility of therapeutically using antibody structures which, by specifically bonding to the receptor substrate or its ligand, in fact interrupt the signal transmission system and inactivate the related intracellular transcription: first, monoclonal antibodies were obtained of chimeric type, i.e. with a portion of the immunoglobulin that preserves the natural murine sequence, such as the sequence known with the name Infliximab. Then, the therapeutic model was perfected with the creation of antibodies with the murine portion, such portion identified as responsible for potential immune responses and for the progressive loss of activity tied to the secretion, reduced or absent, of neutralising antibodies by the organism to which they are administered, such as the substances known with the experimental initials CDP 571 and CDP 870, the latter further protected against the spontaneously induced immunocompetent removal by means of an extended surface pegylation, and known commercially with the Cimzia™ mark.

The protein nature of the antibody structures or soluble receptors has nevertheless required selecting injectable formulations as administration method, with clear involvement and exposure of the entire organism to the action antithetical to TNFα effects, with obvious repercussions on the individual's immune mechanisms as well as possible imbalances in the response to infective agents, tumour agents etc.

Moreover, still with regard to the immune responses, the presence of small quantities of exogenous proteins in circulation can give rise to the secretory stimulus of specific antibodies for each of these, with the clear risk of diminishing the effectiveness of the administered doses, and thus requiring a progressive increase of the doses over time.

The possibility of limiting the anti-TNFα effects only to some organs, or anatomic parts, where the pathological state and/or inflammatory state is confined, would instead be of considerable aid for the resolution of pathologies which are fed by an accentuated secretion of these cytokines: local administration, in fact, would not induce serious perturbations on overall immune mechanisms, hence the individual to whom they are administered retains his typical anti-infective or immune reaction capacities unaltered.

This is the case, for example, of some inflammatory pathologies, limited to well-defined intestinal sectors, such as Crohn's disease or ulcerative colitis, which could receive significant advantages from the administration of substances capable of blocking the intracellular response to TNFα, but which in reality are not treated with these drug types except in the most serious cases, due to the potential risks associated with the administration of these substances.

There is an experimental demonstration of that affirmed: Biancone et al. published a work on Gastrointestinal Endoscopy, 63, 486-492, 2006 in which it is shown how doses of Infliximab about 5 times less than the doses required systemically were effectively utilised with local microinjection techniques, during colonoscopy in patients already surgically operated for Crohn's disease. None of the patients showed the presence of the typical side effects which typically accompany the injectable administration of this antibody, while the size of the lesions was reduced in a consistent manner, proportional to the dose.

It should be underlined that already several of the current anti-TNFα drugs present on the pharmaceutical market have a specific indication for the treatment of intestinal inflammatory pathologies, Ulcerative Colitis and Crohn's Disease, and that all these substances are today systemically administered via injection, specifically by slow infusion, such as Infliximab (Remicade™, Centocor) or subcutaneously, like Certolizumab (Cimzia™, UCB); the latter already has an important advantage, i.e. it can be administered at the patient's home, without having to go to the day hospital for administration, as instead occurs for Remicade.

The therapeutic regimen suggested for Cimzia is 400 mg every 4 weeks, while for Remicade the preferred scheme is one 5 mg/kg dose every 6-8 weeks: as one can see, the administration regimen is pulsated to limit the immune response danger, so to not aggravate the already critical conditions of these patients with serious side effects.

Injectable administration is justified by the fact that these substances are sensitive to the proteolytic action of the digestive enzymes and therefore an oral administration with the traditional formulations could lead to a massive degeneration of the administrated substance and the formation of ineffective if not toxic peptide fragments.

It is known in fact that the stomach and intestine abound with enzymes assigned to break up the elements composing our food into simple elements, such as simple carbohydrates or amino acids, which can be absorbed in the blood for the subsequent transport to the deposit sites and/or elimination sites or to a redistribution inside the organism itself. These enzymes are known with the name of pepsin, trypsin, chymotrypsin etc. and their distribution in the digestive canal has a negative concentration gradient from the stomach to the rectum, i.e. they are more abundant where food is present in more abundance and thus where their action is more greatly required.

Due to their presence, a possible oral administration of protein or peptide substances generally does not produce the desired effects, since a chain of degradative reactions is immediately established which leads to the demolition of the substance in a very short time period; hence the invasive administration forms, such as those injectable, are the elected administration path when one wishes to put into circulation a defined quantity of protein nature substances.

It was surprisingly found recently that the use of a particular administration form, characterised by the presence of substances capable of protecting the protein substance during its gastro-intestinal transit and by a subsequent and preferably progressive liberation of the transported substance during the transit in the final intestinal tract, permits the use of the oral or rectal path to administrate substances even of protein or peptide type.

Employing a site-specific and/or controlled release formulation capable of choosing the colon or rectal intestinal tract as target lends itself to this particular type of therapeutically useful transport.

In the scope of the oral administration path, the pharmaceutical repertory proposes several formulation technologies which claim a predominant colon destination of the transported substance, obtained with techniques which utilise different release mechanisms, such as diffusion, osmosis, swelling and still other release mechanisms.

Among these, a multi-matrix technology is highlighted for the clinical and kinetic effectiveness demonstrated with several already-tested active substances (see Aliment. Pharmacol. Ther., 17, 395-402, 2003). Such technology is composed of a sequential series of different material matrices, including lipophilic substances and hydrophilic polymers, as described in EP 1,183,014.

Forming the object of the present invention is therefore the oral administration of peptide or protein substances by means of a controlled release oral administration technology capable of passing beyond the hostile environment present in the upper digestive sector (stomach, small intestine) and selectively liberating the active principle in the colon and/or rectal part of the intestine.

Also forming the object of the present invention is the localised, site-specific rectal administration of peptides and proteins through liquid or solid administration forms capable of making the transported substance reach the established intestinal site entirely integral.

In some pathological situations, a further therapeutic need is that of ensuring that the release of the active principle occurs in a protected manner within a certain time period, avoiding local concentration peaks of the active principle.

Forming a further object of the present invention is therefore the oral or rectal administration of protein substances in protracted form according to a controlled dissolution profile, so that it takes place in a time not less than a pre-established interval, in such a manner avoiding that peaks are reached while assuring a predetermined concentration level of the released substance in the anatomically affected zone.

Forming a further object of the present invention is the use of a specific technology for the colon release of the drugs, in a pharmaceutical form intended for the oral or rectal administration of proteins, protein fragments, antibody fragments, cytokines, chemokines, anti-cytokine and anti-chemokine substances, peptides, amino acids or other substances mainly composed of amino acids in sequence for the care of pathologies of inflammatory, immunocompetent or tumour nature.

Forming a further object of the present invention is the use of a composition for controlled release oral administration and/or site-specific rectal administration for the colon, in order to administer cytokines or substances capable of modulating the concentration of said cytokines at a local or systemic level.

Finally, forming an object of the present invention is a composition capable of ensuring the oral or rectal administration of an anti-TNFα antibody protein fragment or protein to be used in the care of the intestinal inflammatory pathologies, such as ulcerative colitis, Crohn's disease or celiac disease.

In a typical application of the present invention, a protein peptide substance is used for the manufacture of controlled release capsules, tablets, granules or pellets, formulated in a manner such to protect the transported substance from contact with the digestive enzymes present in the stomach and small intestine and to subsequently release the same substance in a progressive manner along the entire residual intestine tract. In such event, a large portion of the transported protein substance is capable of reaching the cell layer which delimits the colon intestinal lumen and interacting with the cell receptors present therein, also due to the relaxation of the epithelial structure and to the lymphocyte infiltration determined by the existing inflammatory state.

In a further application, a peptide or protein substance is used for producing enemas, foams, suppositories, powders or other suitable forms for site-specific rectal administration, preferably in the distal zone of the digestive tube.

According to the present invention, by powders it is intended a powder to be reconstituted in enema form by the addition of a precise solvent volume, preferably selected from among an aqueous solution buffered to physiological pH or a physiological solution with added substances inhibiting proteolysis, such as ethylenediaminetetraacetic acid or its salts or ionic or non-ionic surface-active agents.

In a further application of the present invention, a peptide or protein substance is used for making enemas or other compositions suitable for rectal administration. Preferably, said enema compositions suitable for rectal administration are formulated to release the drug in a site-specific, modified or controlled manner by the bio-adhesive characteristics of the vehicle. In a preferred application of the invention, an anti-TNFα antibody, possibly found commercially as injectable lyophilised powder, is inserted in a tablet formulation which provides for the presence of lipophilic substances such as waxes of stearic acid, amphiphiles, such as lecithin or other ionic or non-ionic surface-active agents, and hydrophiles, such as cellulose, alkylcellulose or vinylpolymer derivatives, with a sequential matrix structure capable of protecting the active principle from a rapid liberation following oral administration. The tablets are further protected from the acidity of the stomach with acrylic and/or methacrylic copolymers, which have shown to be resistant to the low pH typical of the stomach and make the start of the controlled dissolution independent of the gastric emptying time.

As already mentioned, the technology for making such tablets can be that described in EP 1.183.014, since, as described in the following examples, such technology has been found to be capable of safeguarding the chemical integrity of the protein or peptide substances.

In another application, an anti-TNFα antibody, possibly found commercially as injectable lyophilised powder, is inserted in an enema formulation capable of distributing the active principle along the lumen of the descending colon or sigmoid colon where it can interact with the cell receptors present at the level of the lamina propria.

In another typical application, an anti-TNFα antibody, possibly found commercially as injectable lyophilised powder, is dissolved in a quantity of physiological solution and other auxiliary substances suitable for the preparation of an enema formulation capable of distributing the active principle and keeping it in contact for a long time with the descending or sigmoid colon wall, where the antibody can interact with the TNFα, preventing its binding with the receptors of the cells present at the level of the lamina propria.

In a further application, a protein substance such as an anti-TNFα antibody, possibly found commercially as injectable lyophilised powder, is dispersed in a quantity of tryglicerides at the melting point in the range of 36-38° C., possibly in association with other auxiliary substances suitable for the preparation of a suppository formulation which is capable of distributing the active principle along the mucous of the rectal ampulla and/or sigmoid colon where the antibody can interact with the TNFα, preventing its binding with the receptors of the cells present at the level of the lamina propria.

A further object of the present invention is the use of substances of protein or peptide nature which act as agonists and/or antagonists of the cytokines and/or interleukines and/or growth factors and/or interferons and/or tumour necrosis factors for the preparation of a medication for the localised topical treatment of inflammatory pathologies of the colon or rectal intestinal tract, preferably ulcerative colitis, Crohn's disease, celiac disease or intestinal tumours.

The examples described below make the significance of the invention more appreciable, without however constituting any limitation thereof.

### EXAMPLES

1. A bottle of lyophilised powder containing 100 mg of anti-TNFα antibody dispersed in 500 mg of inert support (Remicade™) was mixed for 5 minutes with 20 mg of soy lecithin, 20 mg of stearic acid, 580 mg of lactose and 500 mg of dibasic calcium phosphate. To the homogenous mixture thus formed, the following were added: a further 2 g of lactose, 500 mg of dibasic calcium phosphate, 40 mg of colloidal silica, 100 mg of methylhydroxypropylcellulose and 40 mg of magnesium stearate before mixing again for 10 minutes. The mixture was subjected to compression on an automatic machine, using punches of 8 mm diameter and obtaining tablets with unit weight of 220 mg, individually containing 5 mg of anti-TNFα antibody. The tablets were then film-coated in a coating pan with an alcoholic mixture of acrylic and methacrylic copolymers (9.6 mg/tablet), triethylcitrate (1 mg/tablet) and with the addition of 4 mg/tablet of talc. The film-coated tablets thus obtained resulted resistant to the artificial gastric juice resistance test at pH 1 for 2 hours, in accordance to the pharmacopoeia requirements for intestinal release tablets. Moreover, in a buffer simulating the intestinal fluid at pH 7.2, the tablets show progressive structural erosion, which is completed in a 6 hour time span.

2. A quantity of lyophilised powder coming from a drug on the market, containing 1000 mg of anti-TNFα antibody dispersed in 5 g of inert support (Remicade™), was mixed for 5 minutes with 200 mg of soy lecithin, 200 mg of stearic acid, 6 g of lactose and 5 g of dibasic calcium phosphate. To the homogenous mixture thus formed, the following were added: a further 20 g of lactose, 5 g of dibasic calcium phosphate, before granulating with the aid of small quantity of aqueous solution containing 1 g of methylhydroxypropylcellulose. The moist mixture was subjected to drying in a low-temperature ventilated oven for one night before adding 300 mg of colloidal silica and 300 mg of magnesium stearate and mixing again for 10 minutes. The mixture was subjected to compression on an automatic machine, using punches of 8 mm diameter and obtaining tablets with 220 mg unit weight, individually containing 5 mg of anti-TNFα antibody. The tablets were then film-coated in a coating pan with an alcoholic mixture of acrylic and methacrylic copolymers (9.6 mg/tablet), triethylcitrate (1 mg/tablet) and with the addition of 4 mg/tablet of talc. The film-coated tablets thus obtained resulted resistant to the artificial gastric juice resistance test at pH 1 for 2 hours, in accordance to the pharmacopoeia requirements for intestinal release tablets, and they showed progressive erosion which lasts at least 5 hours when immersed in a buffer simulating the intestinal pH of 7.2.

3. Using the tablets described in example 1, in order to demonstrate the integrity of the antibody structure and the persistence of its in vivo functionality after the thermal and mechanical stress from the production process, a biological activity test was carried out on human cell cultures incubated with TNFα. In the presence of anti-TNFα antibodies, in fact, the cells were protected, with a survival directly proportional to the antibody quantity. The test was carried out at different concentrations of anti-TNF antibody, so to obtain a protection curve tending towards the theoretical value of the antibody itself administered as a control, using the injectable solution reconstituted from a commercial bottle. The test demonstrated that antiTNF antibody concentrations, obtained by grinding the tablets and drawing a suitable quantity to be added to the culture cells, produce an equivalent quantitative and qualitative protection of the culture cells with respect to that obtainable from the administration of an equal quantity of antibody coming from the solution reconstituted from the commercial Remicade® bottle. This test unequivocally demonstrates that the insertion of the anti-TNF antibody inside the tablet does not cause the total or partial destruction of the protein structure nor diminishes its functional effectiveness towards the target receptor.

4. Using the powder coming from a commercial drug, constituted by a soluble receptor of TNFα and known as Etanercept, tablets have been formulated according to the following procedure: in addition to 500 mg of drug, 150 mg of stearic acid, 270 mg of soy lecithin, 10 g of monohydrate lactose and 20 g of microcrystalline cellulose were added. After having homogenised the powder with an accurate mixing in a small container, the following were added: 5 g of low-viscosity hydroxypropyl methylcellulose and 3.4 g of high-viscosity hydroxypropyl methylcellulose, 200 mg of magnesium stearate and 500 mg of colloidal silica and the mixture was compressed with a compressor machine to the unit weight of about 300 mg. The tablets obtained showed structure persistence with progressive erosion when immersed in simulated intestinal juice at pH 7.2 for over 6 hours. The tablets described above, film-coated with the same film-coating composition based on acrylic and methacrylic copolymers described in example 1, were shown to be resistant to disaggregation in simulated gastric juice at pH 1 for two hours, as provided for by the monographs of the gastro-resistant tablets. The fluid obtained from the dissolution of the tablets, added with suitable excipients, constituted the base for the preparation of a solution to be rectally dropped, by means of a capillary tube of 3.5 cm length, in mice previously treated with dinitrobenzene to induce the presence of ulcers and necrosis of the intestinal mucous, according to a classic pre-clinical model for studies related to experimental colitis. The obtained results, described in the table below, confirm the possibility to induce a dose-correlated improvement or remission of the intestinal inflammatory manifestations following topical intestinal administration of protein nature substances, passing beyond the degradative phenomena induced by the proteolytic enzymes, which are present in this anatomic region in a reduced amount with respect to the remaining portion of the digestive tube.

| Dose | Area of damaged | Reduction (%) |
|---|---|---|
| [mg/animal] | mucous (mm 2) | vs. control |
| 0 (control) | 66 | — |
| 0.0008 | 64 | 3 |
| 0.008 | 29 | 56 |
| 0.08 | 50 | 24 |

5. Using the powder coming from a commercial drug, constituted by a soluble receptor of TNFα and known as Etanercept, tablets were formulated according to the following procedure: in addition to 500 mg of drug, 150 mg of stearic acid, 270 mg of soy lecithin, 10 g of monohydrate lactose and 20 g of microcrystalline cellulose were inserted. After having homogenised the powder with an accurate mixing in a small container, the following were added: 5 g of low-viscosity hydroxypropyl methylcellulose and 3.4 g of high-viscosity hydroxypropyl methylcellulose, 200 mg of magnesium stearate and 500 mg of colloidal silica and the mixture was compressed with a compressor machine to the unit weight of about 300 mg. The tablets obtained showed structure persistence with progressive erosion when immersed in simulated intestinal juice at pH 7.2 for over 6 hours. The tablets described above, film-coated with the same film-coating composition based on acrylic and methacrylic copolymers described in example 1, were shown to be resistant to disaggregation in simulated gastric juice at pH 1 for two hours, as provided for by the monographs of the gastro-resistant tablets. The fluid obtained from the dissolution of the tablets, added with suitable excipients, constituted the base for the preparation of a solution to be rectally dropped, by means of a capillary tube of 3.5 cm length, in mice previously treated with dinitrobenzene to induce the presence of ulcers and necrosis of the intestinal mucous, according to a classic pre-clinical model for studies related to experimental colitis. The obtained results, described in the table below, confirm the possibility to induce a dose-correlated improvement or remission of the intestinal inflammatory manifestations following topical intestinal administration of protein nature substances, passing beyond the degradative phenomena induced by the proteolytic enzymes, which are present in this anatomic region in a reduced amount with respect to the remaining portion of the digestive tube.

| Dose | Area of damaged | Reduction (%) |
|---|---|---|
| [mg/animal] | mucous (mm 2) | vs. control |
| 0 (control) | 62 | — |
| 0.0008 | 61 | 2 |
| 0.008 | 32 | 49 |
| 0.08 | 51 | 19 |

6. A quantity of lyophilised powder coming from a commercial drug, containing 1000 mg of anti-TNFα antibody dispersed in 5 g of inert support (Remicade™), was brought into solution with a phosphate buffer added with isotonising and surface-active agent, until reaching a concentration in the range of 0.5-5 mg/ml. The solution was then used as an enema for the rectal administration of the antibody.

7. A quantity of lyophilised powder coming from 5 bottles of a commercial drug, containing 100 mg/bottle of anti-TNFα antibody dispersed in an inert support (Remicade™), was dispersed in a structured vehicle formed by a hydrophilic polymer dispersed in an isotonic phosphate buffer added with a small quantity of non-ionic surface-active agent, polysorbate 80, until a gelatinous consistency is attained along with a n active principle concentration in the range of 1-10 mg/g. The gel thus obtained was used in a 0.2 g dose for a rectal administration in a rat in which experimental colitis has been induced by means of dinitrobenzene administration. The administration, repeated for three days, produced a consistent and dose-proportional reduction of the area of DNB-caused ulceration, according to the following table:

| Dose | Area of damaged | Reduction (%) |
|---|---|---|
| (mg/animal) | mucous | vs. control |
| 0 (control) | 262.35 | — |
| 0.008 | 86.27 | 66.7 |
| 0.2 | 136.46 | 48.0 |
| 5 | 181.95 | 30.6 |

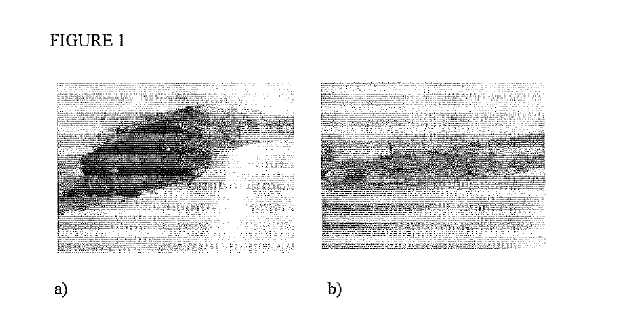

In FIG. 1, an example is shown of necrotised area (a) and regenerated area after treatment with the formulation of example 6 (b).

8. A bottle of lyophilised powder containing 100 mg of anti-TNFα antibody dispersed in an inert support (Remicade™) was mixed for 5 minutes with 20 mg of soy lecithin, 20 mg of stearic acid, 580 mg of lactose and 500 mg of dibasic phosphate calcium. To the homogenous mixture thus formed, the following were added: a further 2 g of lactose, 500 mg of dibasic phosphate calcium, 40 mg of colloidal silica, 100 mg of methylhydroxypropylcellulose and 40 mg of magnesium stearate before mixing again for 10 minutes. The mixture was subjected to compression on an automatic machine, using punches of 8 mm diameter and obtaining tablets of 220 mg unit weight, individually containing 5 mg of anti-TNFα antibody. The tablets were then broken up in a mortar until less then 0.5 mm granule size. The granules thus obtained were dispersed in an isotonic vehicle based on saline phosphate buffer, added with a small quantity of surface-active substance so to favour its wettability and utilised for rectal administration in test rats in a DNB-induced experimental colitis test; after three days the rats showed a consistent diminution of the necrosis area produced by the preceding intracolon administration of nitrobenzene substance.

1. A pharmaceutical composition for the oral or rectal administration of active principles, characterised in that said active principles are substances of protein or peptide nature which act as agonists and/or antagonists of cytokines and/or interleukines and/or growth factors and/or interferons and/or tumour necrosis factors, and that said substances are formulated in the form of tablets, capsules, granules, pellets, enemas, suppositories, foams or powders with auxiliary substances suitable for ensuring a release of the active principle in the intestine, preferably in the colon or rectum.

2. The pharmaceutical composition of claim 1, characterised in that said substances are adapted to act locally at the intestinal level by blocking the specific receptors for these substances or by interacting directly with the circulating cytokines and limiting their availability for the intestinal tissue receptors.

3. The pharmaceutical composition of claim 1, characterised in that said active principles are peptide or protein substances which have been specifically isolated or synthesised to block or reduce the TNFα activity on the intestinal tissue cells.

4. The pharmaceutical composition of claim 1, characterised in that said active principles belong to the chemical class of the specific monoclonal antibodies and/or polyclonal antibodies and/or soluble receptors for cytokines, in particular for TNFα and TNFβ, and/or by antibody portions containing at least one sequence portion of the variable region of an immunoglobulin capable of binding to TNFα and TNFβ and/or by sequence portions of the cell receptor for TNFα and TNFβ and/or by analogous structures of such variable regions of anti-TNFα and TNFβ antibodies.

5. The pharmaceutical composition of claim 1, characterised in that said active principles are of partial or total murine, chimeric or human nature.

6. The pharmaceutical composition of claim 1, characterised in that the pharmaceutical administration form is constituted by controlled-release and gastro-protected tablets or capsules containing modified-release and gastro-protected mini-matrices or granules.

7. The pharmaceutical composition of claim 1, characterised in that the pharmaceutical form is constituted by multimatrix granules or tablets, in which at least a hydrophilic matrix and a lipophilic and/or amphiphic matrix are co-present.

8. A multimatrix solid composition for the intestinal release oral administration of one or more monoclonal or polyclonal antibodies or soluble receptors, having specific blocking functions of the circulating excess of cytokines and/or TNFs for the intestinal cells, aimed for the cure of pathologies of autoimmune, inflammatory or tumour nature, such as Ulcerative Colitis, Crohn's disease, celiac disease or colorectal cancer.

9. The pharmaceutical composition of claim 1, characterised in that the active principle is constituted by an association of monoclonal or polyclonal antibodies or by soluble receptors for TNF in association with other active principles with immunomodulating or anti-inflammatory or chemotherapy action.

10. A method for treating an inflammatory condition of the rectum or colon comprising the administration of the pharmaceutical composition of claim 1.

11. The method of claim 10, wherein said inflammatory condition is selected from ulcerative colitis, Crohn's disease, celiac disease and intestinal tumours.

12. The method of claim 10, wherein the pharmaceutical composition is in the form of a tablet, capsule, granule or pellet.

13. The method of claim 10, wherein the pharmaceutical composition is in the form of an enema, foam, suppository or powder.
